(19) United States
(12) Patent Application Publication
(10) Pub. No.: US 2024/0000997 A1
(43) Pub. Date: Jan. 4, 2024

(54) USE OF SCLERAL OSSICLES INTEGRATED IN 3D RETICLES FOR THE RECOVERY OF BONE INJURIES OF CRITICAL SIZE IN THE HEALTHCARE AND VETERINARY SECTOR

(71) Applicant: UNIVERSITA' DEGLI STUDI DI MODENA E REGGIO EMILIA, Modena (IT)

(72) Inventor: Carla Palumbo, Modena (IT)

(21) Appl. No.: 18/036,368

(22) PCT Filed: Nov. 4, 2021
(86) PCT No.: PCT/IT2021/050362

§ 371 Date: May 10, 2023

(30) Foreign Application Priority Data
Nov. 24, 2020 (IT) 102020000028229

(51) Int. Cl. A61L 27/36

(52) U.S. Cl. CPC A61L 27/3608; A61L 2430/02

(57) ABSTRACT

Scleral ossicles (OS) are described, integrated in 3D lattices, to be used as a naturally decellularized material for the regeneration of at least a portion of a bone system, in the veterinary and healthcare fields.

### RELATED APPLICATIONS

This application is the national stage under 35 U.S.C. 371 of International Application No. PCT/IT2021/050362, filed Nov. 4, 2021; which claims priority to Italian Application No. 102020000028229, filed Nov. 24, 2020; the entire contents of each of which are incorporated by reference in this application.

### FIELD OF THE INVENTION

The present invention relates to the use of scleral ossicles integrated in 3D lattices for the recovery of bone lesions of critical size in the healthcare and veterinary field. Furthermore, the present invention relates to a naturally decellularized material consisting of scleral ossicles for the regeneration of bone tissue.

### BACKGROUND OF THE INVENTION

The present invention relates to natural bio-materials, which can be integrated with 3D lattices or other supports, for the recovery of bone lesions of a critical size in the veterinary and sanitary field.

In general, the present invention relates to materials for grafts or devices for tissue regeneration.

In particular, the present invention refers to composite materials, i.e. containing a material dispersed in a matrix of the same or different material; materials containing ingredients of indeterminate constitution or their reaction products, e.g. graft tissue, natural bone, extracellular matrix, demineralized bone matrix [DBM], bone powder.

At present there are no patents that can be superimposed on the proposed one. Patent application WO 2018/191274 A1 relates to a tissue generation system comprising: a plurality of modular MTFU tissue forming units comprising a plurality of biocompatible scaffolding particles seeded with a plurality of exogenous cells capable of forming at least one type of tissue needed to repair a tissue defect, in which cells are initially coupled to the scaffolding particles; a perfusion fluid delivery conduit adapted to deliver the medium to a tissue defect packed with MTFUs; and an NPWT negative pressure wound therapy system comprising an NPWD negative pressure wound dressing coupled to a SAP sub-atmospheric pressure device such that when the NPWD is fixed on the defect of the tissue packed with MTFUs, the SAP device and the NPWD function by pulling the media through the gaps between the packed MTFUs within the tissue defect to perfuse the cells in the MTFUs with the media.

From data collected in the last 10 years on the characteristics of the scleral ossicles, the idea was born of proposing them as an integral part to be introduced into three-dimensional lattices (or other support) for the non-spontaneous recovery of bone lesions, regardless of the nature and geometry of the reticulum, and the size of the bone lesion to be recovered.

At present, the use of scleral ossicles for bone regeneration does not appear to be found in the patent state of the art.

To make a comparison, since similar patents do not exist, patent application WO 2018/191274 A1 represents a very different and rather complicated inventive idea in the constitution of its parts/phases, implying so-called modular tissue forming units which contain cells seeded on portions of scaffolds, referred to as modular scaffolding particles, positioned and perfused in the lesion site inducing, with microfluidics, a negative pressure to trigger the repair of damage. Therefore, the system of patent application WO 2018/191274 A1 is totally different from the object of the present invention, also providing for the use of seeded cells and implying the cell therapy procedures completely absent in the present invention, with the only similarity in the tissue regeneration target.

In the current invention, it is conceptually emphasized that the application of scleral ossicles is independent from the chemical nature, polymers, nanoparticles, etc., and the geometry, latticework with different design and porosity, of the 3D lattice that hosts them.

### SUMMARY OF THE INVENTION

Object of the present invention is solving the aforesaid prior art problems by using the scleral ossicles, OS, for the regeneration of at least a portion of a bone system.

A further object is providing a perfectly biocompatible material without antigenic properties for the regeneration of at least a portion of a bone system.

A further object is providing a method of preparing scleral ossicles in order to be able to use the scleral ossicles (OS) for the regeneration of at least a portion of a bone system.

The aforementioned and other objects and advantages of the invention, which will emerge from the following description, are achieved through the use of scleral ossicles (OS) for the regeneration of bone tissue as claimed in the respective independent claim.

Furthermore, the aforementioned and other objects and advantages of the invention, which will emerge from the following description, are achieved with a naturally decellularized material consisting of scleral ossicles for the regeneration of bone tissue as claimed in the respective independent claim.

Preferred embodiments and non-trivial variants of the present invention form the subject of the dependent claims.

It is understood that all attached claims form an integral part of the present description.

It will be immediately obvious that innumerable variations and modifications (for example relating to shape, dimensions, arrangements and parts with equivalent functionality) can be made to what is described without departing from the scope of the invention as appears from the attached claims.

The proposal to use scleral ossicles (OS) for the regeneration of critical skeletal lesions, integrating them with three-dimensional lattices, regardless of the composition and geometry of the lattices, is innovative in that, unlike other proposals, it does not foresee the use of cells at all, stem cells, mesenchymal cells, etc., and therefore does not fall within the methods of cell therapy which may involve collateral risks. The innovative idea is to use a naturally decellularized material present and available in nature at no cost for regenerative strategies, i.e. scleral ossicles (OS), perfectly biocompatible and devoid of antigenic properties and which, above all, does not require particular manipulations to be used. It is also a versatile natural biomaterial, which can be introduced into any type of support network which may have specific characteristics ad hoc for the type of lesion or which may be standard and with different volumes and, depending on the extent of the lesion to be repaired, present a number different slits (each comprising a single OS) capable of accommodating an adequate number of scleral ossicles.

### BRIEF DESCRIPTION OF THE DRAWINGS

The present invention will be better described by some preferred embodiments, provided by way of non-limiting example, with reference to the attached drawings, in which:

FIG. 1, 2, 3 show a plurality of scleral ossicles (OS) used for the regeneration of a bone system according to the present invention;

FIG. 4, 5, 6 show an orthogonal projection view of a scleral ossicle of the plurality of scleral ossicles OS of the previous figures;

FIG. 7 shows a diagram of the manufacturing steps of the naturally decellularized material consisting of at least one scleral ossicle (OS);

FIG. 8 schematically shows a component into which the naturally decellularized material object of the present invention can be introduced; and

FIG. 9 schematically shows the naturally decellularized material object of the present invention, important for triggering tissue regeneration if introduced into support material.

### DETAILED DESCRIPTION OF PREFERRED EMBODIMENTS

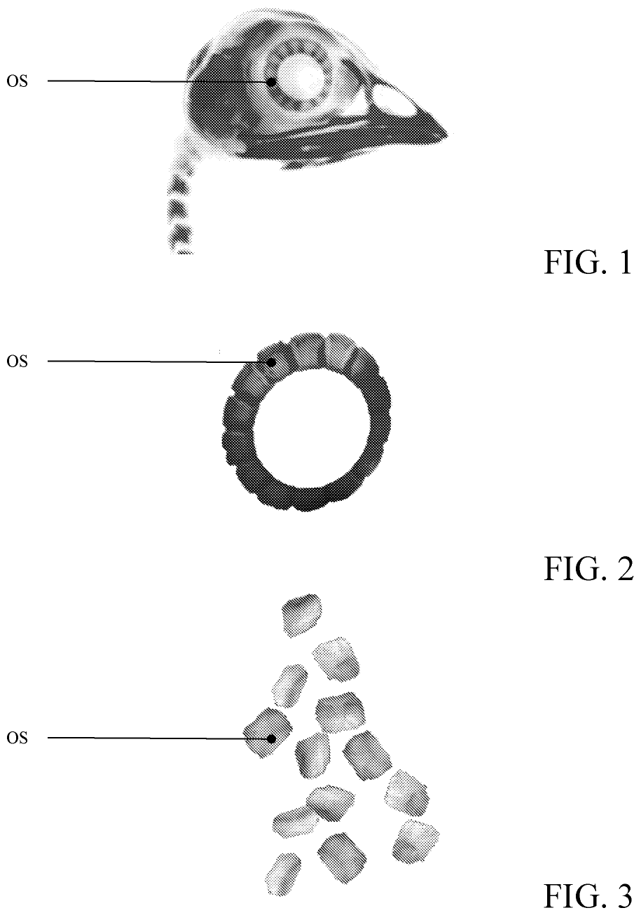

Referring to FIG. 1 to 6, it is possible to note how to obtain the OS scleral ossicles to allow the regeneration of at least a portion of a bone system, in the veterinary and health care field.

In particular, the OS scleral ossicles allow to activate cellular colonization and differentiation processes, aimed at tissue regeneration, with the aim of obtaining results that without the OS scleral ossicles would be obtainable in a quantitatively lesser measure and with longer times.

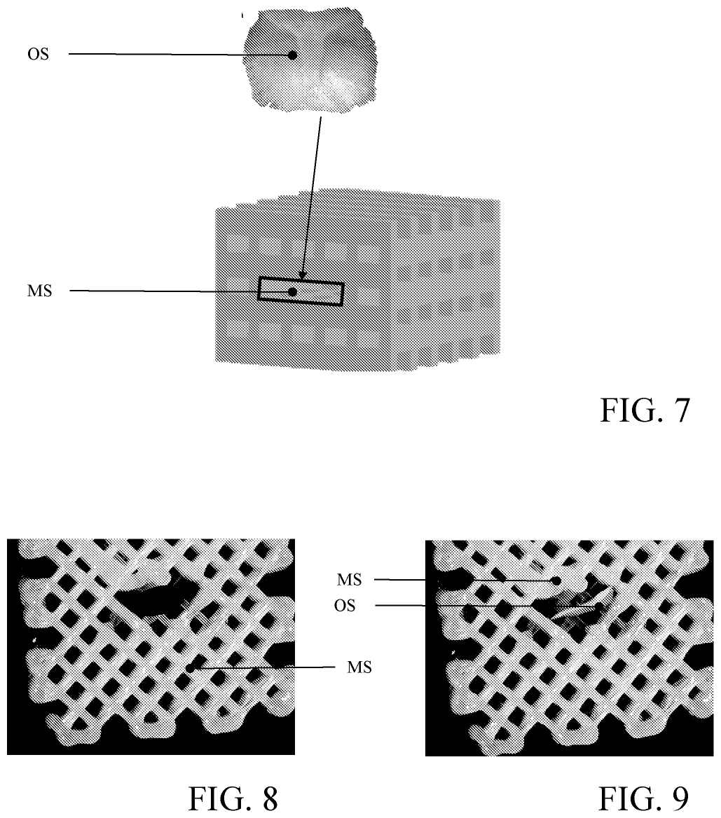

Referring to FIG. 7, 8, 9, it is possible to note that a naturally decellularized material for the regeneration of at least a portion of a bone system consists of at least one scleral ossicle OS.

In particular, a scleral ossicle OS introduced into an MS support of various nature and geometry that fills the volume of a bone lesion, allows inducing the endothelial and osteoblastic cells of an injured segment to migrate and differentiate into the MS support.

In such a material, the MS support is equipped with at least one cavity capable of accommodating the scleral ossicles OS.

Preferably, such at least one scleral ossicle OS is that of birds of the galliform family, in particular chickens.

In particular, the naturally decellularized material allows the activation of cellular colonization and differentiation processes, aimed at tissue regeneration, with the aim of obtaining results that without the naturally decellularized material would be obtainable in a smaller quantity and with longer times.

A method of preparing scleral ossicles for the use of OS scleral ossicles for regeneration of at least a portion of a bone system is configured to obtain a plurality of OS scleral ossicles disarticulated from the sutures present at the sclero-corneal border of the eyeball, deprived of the soft tissues that surround them, such as periosteum, sterilized with UV.

### EXAMPLES

FIG. 2 shows, by way of schematic example, the site of sampling of scleral ossicles (OS) and FIG. 8 shows a hypothetical 3D-lattice that can accommodate an OS (in a special slot of appropriate size). Obviously, it is possible to devise reticles that have several slits, in each of which only one scleral ossicle is housed.

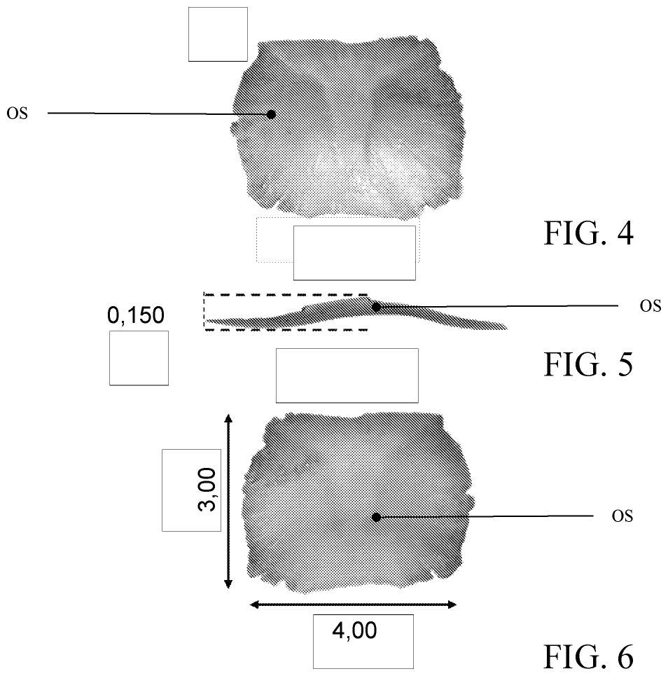

Referring to FIG. 4, 5, 6, the scleral ossicles are very particular and small quadrangular bone segments, about 4 mm×3 mm, with a thickness of about 0.2 mm, naturally decellularized, present in the eyeball of lower vertebrates with protruding eyes, such as birds, reptiles, fish, located at the sclero-corneal border and having the purpose of protecting the eyeball from deformation during flight or swimming.

Due to their functional characteristics, they must not be able to respond, as all other skeletal segments do, to the metabolic and mechanical needs of the organism to which the bone generally responds through a process called remodeling, which involves the continuous erosion and reconstruction of the skeletal segments, in response to the dual metabolic and mechanical needs of the organism. Since bone remodeling is modulated by osteocytes, mature cells that live within the bone matrix, in the scleral ossicles, once the final size is reached, the osteocytes massively undergo apoptosis, programmed death, precisely to avoid the occurrence of remodeling.

Therefore, they are naturally decellularized ossicles.

These first evidences were fundamental, together with the observations reported below, for the conception of the present patent proposal.

In particular, the focus is on the scleral ossicles of the chicken which have been the subject of basic research; they are obtainable at no cost, unlike all other bone substitutes on the market, because they are avian butchery waste. The following characteristics/properties have been studied and demonstrated on these natural biomaterials: absence of cytotoxicity; in vitro biocompatibility; ability to induce angiogenesis and osteogenesis in vitro and in vivo; absence of inflammation following subcutaneous implantation in vivo in non-immunosuppressed animals.

On the basis of these characteristics common to various devices used for bone regeneration and often ad hoc packaged for critical lesions, the idea was born to use them as triggers for angiogenesis and osteogenesis by introducing them into supports of various kinds and geometry that can fill the volume of a bone lesion, in which they can induce the endothelial and osteoblastic cells of the host bone to migrate and differentiate. In particular, the contextual ability to induce angiogenesis and osteogenesis already demonstrated with basic research procedures is not irrelevant, as bone regeneration cannot occur if angiogenesis does not occur first, like what happens in neoplastic processes.

A trellis housing the scleral ossicles can be of any geometry and composition type, as long as it has one or more slots of sufficient size to each accommodate a scleral ossicle, approximately 4 mm×4 mm×0.3 mm.

To use the scleral ossicles for the regeneration of portions of a bone system, a procedure for the treatment of the scleral ossicles consists in making the scleral ossicles: 1—disarticulated from the sutures present at the sclero-corneal border of the eyeball; 2—carefully deprived of all soft tissues, periosteum, surrounding them; 3—UV sterilized, etc.

Thanks to these steps of the scleral ossicles treatment process, it was possible to demonstrate the angiogenic and osteogenic abilities of the scleral ossicles, capacities necessary to use the scleral ossicles for the proposed purposes.

From a translational perspective, we intend to propose the use of scleral ossicles in two areas: i) veterinary, to recover severe bone defects in farm animals, horses, cattle, sheep, pigs, which would be slaughtered following large lesions, inducing large economic losses; ii) sanitary, in humans. It has already been shown that chicken scleral ossicles implanted in the subcutaneous tissue of non-immune-depressed mice do not induce an inflammatory reaction or rejection because, being naturally decellularized, they mostly lose their antigenic properties.

Currently, preliminary studies aimed at demonstrating the immediate applicability of the use of scleral ossicles have already been carried out, as basic research on: [1], evidence of the absence of cells in the scleral ossicles at the end of development; [2], ability of the scleral ossicles to allow differentiation of endothelial and osteoblastic cells close to its surfaces, [3], in vitro and in vivo biocompatibility of scleral ossicles.

Since the invention is conceptually related to the use of the proposed material, already proven suitable for the intended use, no further studies are deemed necessary.

However, pilot experiments were carried out, the data of which are still confidential and unpublished, regarding the introduction of scleral ossicles into 3D supports to allow for a good colonization of the same; these pilot experiments (not yet disclosed) show the ability of OS to implement the colonization of three-dimensional pylons, regardless of their composition and geometry.

1. Scleral ossicles (OS) integrated in 3D lattices to be used as a naturally decellularized material for the regeneration of at least a portion of a bone system, in the veterinary and health sectors.

2. Material according to the preceding claim, characterized in that said at least one scleral ossicle (OS) introduced into a support (MS) of various nature and geometry, which fills the volume of a bone lesion, can induce endothelial and osteoblastic cells of an injured segment to migrate by differentiating into the support (MS).

3. Material according to the preceding claim, characterized in that said support (MS) is provided with at least one cavity capable of accommodating said at least one scleral ossicle (OS).

4. Material according to one of the preceding claims, characterized in that said at least one scleral ossicle (OS) is that of birds of the galliform family, in particular chickens.

5. Use of one or more scleral ossicles as a naturally decellularized material to be introduced into a support (MS) of various nature and geometry, to activate cellular colonization and differentiation processes, aimed at tissue regeneration, with the aim of obtaining results that without said naturally decellularized material would be obtainable in a smaller quantity and with longer times.
